(19) United States
(12) Patent Application Publication
(10) Pub. No.: US 2010/0241031 A1
(43) Pub. Date: Sep. 23, 2010

(54) Analyte Test Device Integral With Lancet Firing Mechanism

(75) Inventor: Siew Kong LAI, Singapore (SG)

Correspondence Address: LAWRENCE Y.D. HO & ASSOCIATES PTE LTD, 30 BIDEFORD ROAD, #02-02, THONGSIA BUILDING, SINGAPORE, 229922, SG

(73) Assignee: Venture Corporation Limited, Singapore (SG)

(21) Appl. No.: 12/407,796

(22) Filed: Mar. 20, 2009

(51) Int. Cl. A61B 5/151

(52) U.S. Cl. 600/583

(57) ABSTRACT

The present invention provides an analyte test device (5) for determining concentration of an analyte in a physiologic fluid. The analyte test device (5) includes a test meter (7) and a lancet device (10, 110). The lancet device (10,110) has an integral lancet (20,120), a lancet casing (40,140) and a test strip (60). The lancet (20,120) is locked onto the lancet casing (40,140) by a catch (32,132). When the lancet device (10) is inserted into a receptacle (R) on the test meter (7), a spigot (Q) unlocks the catch (32) from the lancet casing (40) so that the lancet (20) takes on the characteristic of a probe (P) that is connected to a lancet firing mechanism disposed in the test meter (7). Alternatively, a collar (C) on the probe (P) disengages the catch (132) from the lancet casing (140) when the lancet device (110) is inserted into the test meter (7).

### REFERENCE TO RELATED APPLICATIONS

The present application claims priority to U.S. application Ser. No. 12/327,817 filed on Dec. 4, 2008, entitled “Lancing Mechanism For Mimimizing Pain”, which is incorporated by reference in its entirety.

### FIELD OF INVENTION

The present invention relates to an analyte test meter which has an integral lancet firing mechanism and a disposable lancet-test strip assembly.

### BACKGROUND

Diagnosis of a patient's medical condition, such as blood glucose, is important in health management. Hence, it is desirable for a patient to perform tests routinely, quickly and reproducibly by oneself or by a caregiver outside of a laboratory setting. This may involve inserting a test strip into an analytical meter, puncturing one's finger tip with a separate lancing device to obtain a droplet of blood, transferring the droplet of blood onto a test element on the test strip, and checking a reading on the analytical meter for the concentration of a single analyte in the droplet of blood. The analyte may be blood glucose for a person with diabetes, cholesterol for a person with cardiovascular condition, uric acid for a person with gout, drug for monitoring effect of a therapy or presence of illegal drugs, and so on. Often, such diagnoses are repeated several times in a day and providing a diagnostic tool that is easy to operate and yet giving a less painful, if not a painfree, experience is desired.

U.S. Pat. No. 7,396,334, assigned to Roche Diagnostics Operations, Inc., describes a needle and lancet body integral with a test element. The accompanying figures show the tip of the needle is embedded in an elastic material whilst the drive end of the needle extends from the rear of the lancet body.

US Publication No. 2008/0262386, also assigned to Roche Diagnostics Operations, Inc., describes an analytical system for detecting an analyte in a body fluid, and a disposable integrated puncturing and analyzing element. The instrument is cheap to manufacture and allows a user full control over the individual steps in collecting a blood sample for analysis.

US Publication No. 2008/058631, assigned to Beckton Dickinson, describes a blood glucose meter having integral lancet device and test strip storage vial for single hand use. By combining these multiple components into a single device, the glucose meter requires fewer steps in its use.

Despite development in the art, it can thus be seen that there exists a need for a device and method for analyzing a person's physiologic fluid for a medical condition that overcome the shortcomings of known devices.

### SUMMARY

The following presents a simplified summary to provide a basic understanding of the present invention. This summary is not an extensive overview of the invention, and is not intended to identify key features of the invention. Rather, it is to present some of the inventive concepts of this invention in a generalised form as a prelude to the detailed description that is to follow.

The present invention seeks to provide an analyte test device for determining concentration of an analyte in a physiologic fluid. The analyte test device includes a test meter and a disposable lancet device.

In one embodiment, the analyte test device comprises a test meter and a lancet device. The lancet device has a casing, a lancet and a test strip. The lancet is disposed and locked inside the casing by a catch, whilst the test strip is disposed on said casing. The lancet device is operable to be removeably connected to said test meter via a receptacle on said test meter. The receptacle is operable to engage with said catch to unlock said lancet from said casing when said lancet device is inserted into said receptacle and to disengage said catch to relock said lancet inside said casing when said lancet device being removed from said receptacle.

In another embodiment, the present invention provides a kit for an analyte test device. The kit comprises a lancet, a lancet casing and a test strip. The lancet has a catch, which is operable to lock said lancet inside said lancet casing, and said test strip is operable to be attached onto said lancet casing to form a lancet device.

In yet another embodiment, the present invention provides a method for determining an analyte in a physiologic fluid. The method comprises: supplying a lancet device, said lancet device comprises a lancet, a lancet casing and a test strip; wherein said lancet has a catch that is operable to lock said lancet inside said lancet casing, and said test strip is operable to be attached onto said lancet casing to form a lancet device; inserting the lancet device into a receptacle of a test meter, wherein said catch is then deactivated and the lancet is operable to take on the characteristic of a lancet firing mechanism disposed inside said test meter; collecting a physiologic fluid sample and transferring the fluid sample on an analyte sensing end on said test strip; and obtaining a reading from said test meter.

### BRIEF DESCRIPTION OF THE DRAWINGS

This invention will be described by way of non-limiting embodiments of the present invention, with reference to the accompanying drawings, in which:

FIG. 1 illustrates an analyte test device according to an embodiment of the present invention;

FIG. 2A illustrates a lancet device formed integral with an analyte test strip for use with the analyte test device shown in FIG. 1 in accordance with another embodiment of the present invention;

FIG. 2B illustrates an exploded view of the lancet device;

FIG. 2C illustrates a lancet shown in FIGS. 2A and 2B; and

FIG. 2D illustrates a section view of the lancet device;

FIG. 3 illustrates a part sectional view of the lancet device coupled to a probe and test terminals of an associated analyte test device in accordance with another embodiment of the present invention;

FIG. 4A illustrates a lancet according to another embodiment of the present invention; and FIG. 4B illustrates a lancet device incorporating the lancet shown in FIG. 4A; FIG. 4C illustrates a lancet shown in FIG. 4A; and

FIGS. 5A-5G illustrate a sequence of steps for using the lancet device and associated analyte test device in accordance with another embodiment of the present invention.

### DETAILED DESCRIPTION

One or more specific and alternative embodiments of the present invention will now be described with reference to the attached drawings. It shall be apparent to one skilled in the art, however that this invention may be practised without such specific details. Some of the details may not be described at length so as not to obscure the invention. For ease of reference, common reference numerals or series of numerals will be used throughout the figures when referring to the same or similar features common to the figures.

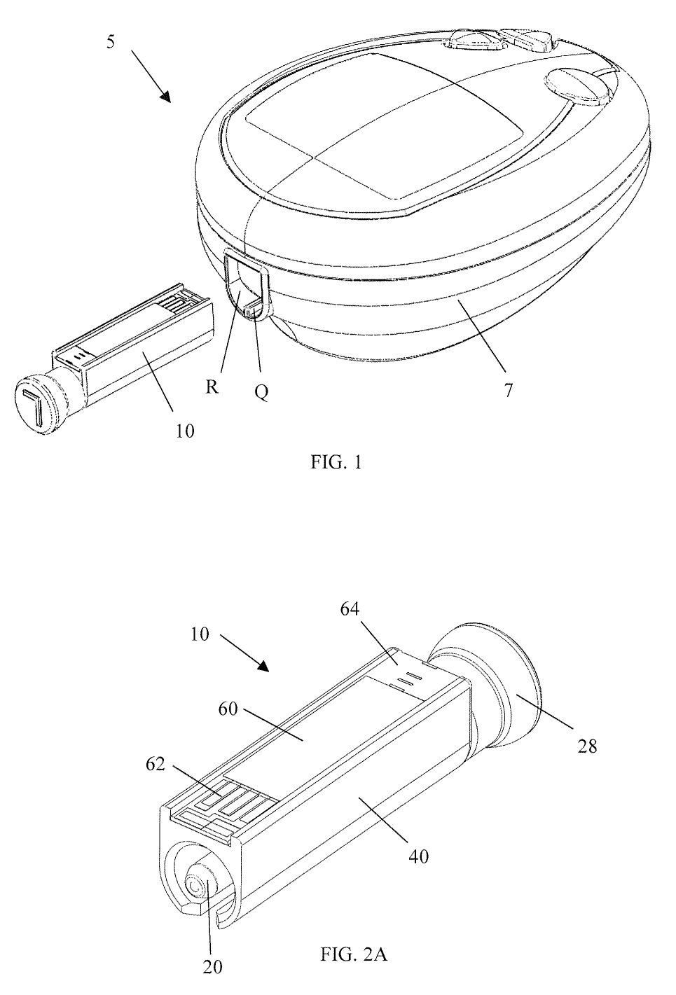

FIG. 1 shows an analyte test device 5 according to an embodiment of the present invention. As shown in FIG. 1, the analyte test device 5 includes a test meter 7 and a removeable lancet device 10.

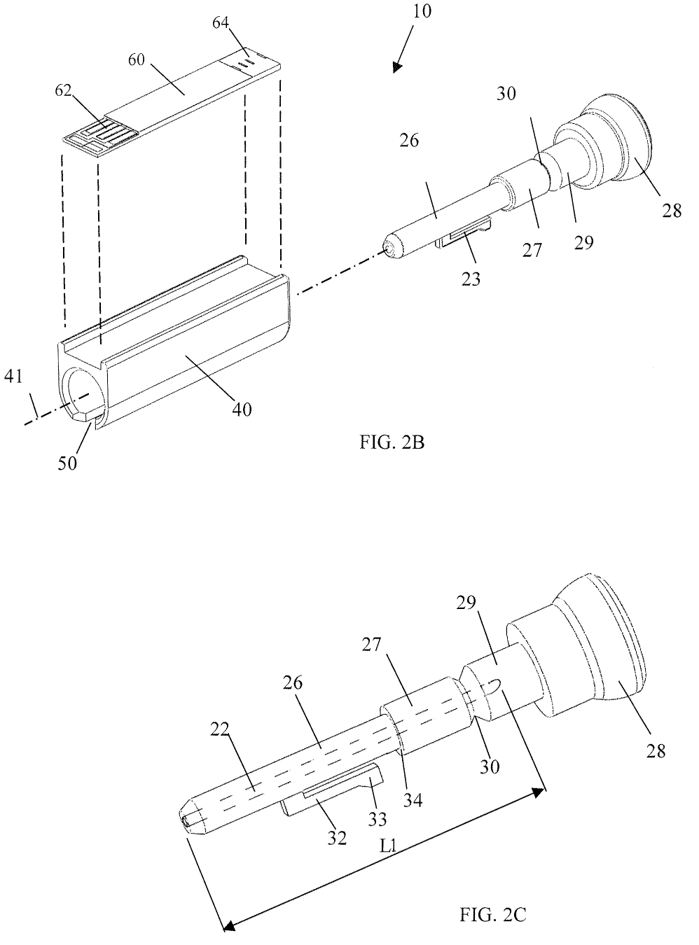

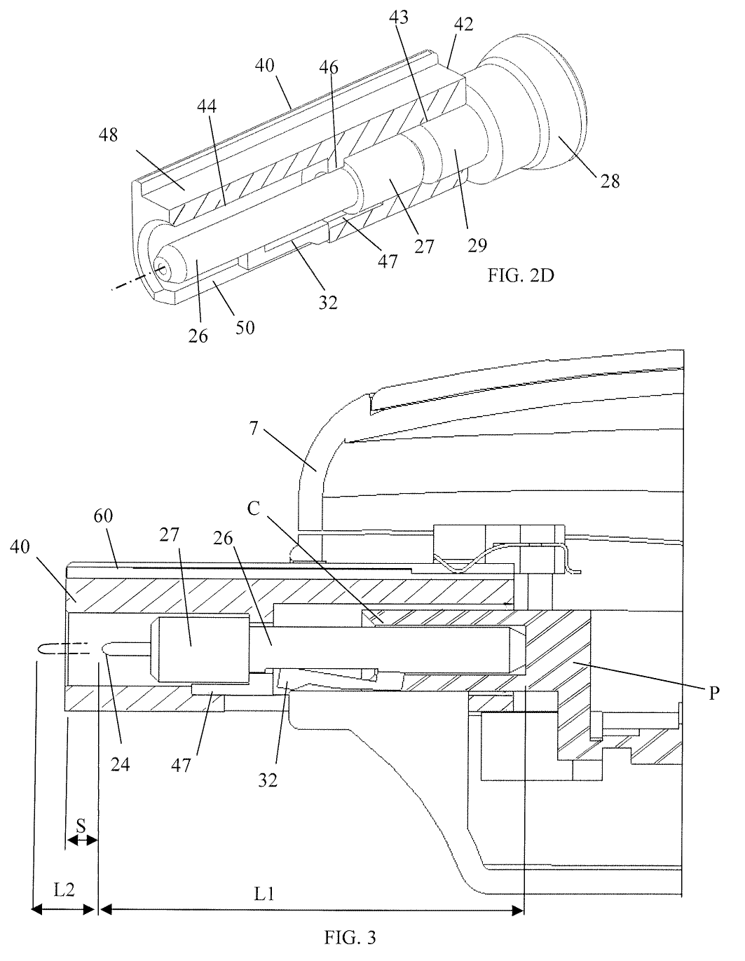

FIG. 2A shows the lancet device 10 according to another embodiment of the present invention. The lancet device 10 is an assembly of a lancet 20, a casing 40 and an analyte test strip 60. As shown in FIG. 2A, the lancet 20 is disposed inside the casing 40 and the test strip 60 is disposed on the casing 40. FIG. 2B shows an exploded view of the lancet device 10. As shown in FIGS. 2A and 2B, each test strip 60 has a terminal end 62 at one end and an analyte sensing end 64 at the opposite end.

FIG. 2C illustrate the lancet 20 shown in FIGS. 2A and 2B. As shown in FIG. 2C, the lancet 20 includes a needle 22 that is sterilized and insert molded with a thermoplastic such that the needle's pointed end 24 is molded within a cap 28. As shown in FIG. 2C, the cap 28 is connected to the molded body of the lancet 20 by a notch 30 of reduced cross-section. In between the cap 28 and the notch 30 is a locating part 29. The locating part 29 is concentric with the needle 22. The molded lancet body is made up of two cylindrical parts 26, 27 with an end of the larger sectional part 27 partly forming the notch 30 and the joint with the smaller sectional part 26 forming a step 34. Projecting from the cylindrical surface of the smaller sectional part 26, there is an L-shaped catch 32. The L-shaped catch 32 has an arm 33 pointing in the same direction as the needle pointed end 24. The free end of the lancet body 26 is chamfered for easier insertion into a collar C at a probe P inside the test meter. In one embodiment, the length from the free end of the lancet body 26 to the needle's pointed end 24 is L1; a corresponding length of the needle's pointed end 24 to a front end or face 42 of the casing 40 is L2. Length L2 is more clearly seen in FIG. 3.

FIG. 2D shows a sectional view of the lancet device 10 shown in FIG. 2A. As shown in FIG. 2D, the casing 40 is elongate and has a longitudinal axis 41 along its length. The casing 40 is hollow and has two cylindrical bores 43,44. A collar 46 separates the two cylindrical bores 43,44. The smaller of the cylindrical bore 43 is at the front end 42 of the casing 40. The cylindrical bore 43 is dimensioned to fit with the locating part 29 of the cap 28 to give an interference fit whilst the fit with the body 27 of the lancet 20 is a clearance fit. The larger of the cylindrical bore 44 is dimensioned to accommodate the collar C of the probe P and the fit between an external dimension of the collar C and the cylindrical bore 44 is also clearance fit. The cylindrical bore 44 has a longitudinal slot 50. The longitudinal slot 50 is dimensioned so that the L-shaped catch 32 on the lancet 20 is slidable in the longitudinal slot 50. In an extension of the longitudinal slot 50 but on the inside of the cylindrical bore 43 is a longitudinal groove 47. The length of the groove 47 is dimensioned so that it is longer than the stroke S of the probe P. As shown in FIGS. 2D and 3, the groove 47 cuts through the collar 46. Diametrally opposite the longitudinal slot 50 is a flat surface 48 on the top of the casing 40 for mounting the analyte test strip 60.

In use, the lancet 20 is disposed in the hollow casing 40 such that the free end of the L-shaped catch 32 engages with the end edge of the longitudinal slot 50 so that the lancet 20 becomes locked onto the casing 40 as one assembly. From FIG. 2D, it is seen that the free end of the L-shaped catch 32 engaging with the end edge of the longitudinal slot 50 causes the step 34 on the lancet 20 to press against the collar 46 on the casing 40. The fit between the collar 46 and the body 26 of the lancet 20 is also clearance fit and the concentricity of the body 26,27 of the lancet 20 with the longitudinal axis 41 is maintained by the interference fit between the locating part 29 of the cap 28 that is journalled in the cylindrical bore 43.

FIG. 3 shows a part sectional view of the lancet device 10 coupled to the probe P of the associated analyte test meter 7 according to an embodiment of the present invention. As shown in FIG. 3, the cap 28, whose outline is indicated by a phantom line, has been sheared off at the notch 30 and the pointed tip 24 of the lancet needle 22 is exposed. The fit between the collar C at the free end the probe P and the cylindrical body 26 of the lancet 20 is an interference fit; this interference fit allows the lancet 20 to be retained in the probe P so that the lancet 20 and probe P move as one body during lancing of the probe P. In addition, this interference fit and the clearance fit around the lancet body 26,27 allow the lancet 20 to take on the characteristic movements of the probe P during lancing. In use, the lancet device 10 is fully inserted into the probe P of an associated analyte test meter 7; in other words, the free end of the cylindrical body 26 of the lancet 20 bottom-out in the collar C when the lancet device 10 is fully inserted into the analyte test meter 7. This bottoming-out of the lancet 20 in the collar C allows a penetration depth of the lancet that is predetermined via a depth penetration mechanism (not shown in FIG. 3) to be determinate. In other words, with an interference fit between the lancet body 26 and the collar C of the probe, there is no slipping of the lancet from the collar C and therefore the amount of travel of the needle pointed end 24 into a user's skin is substantially determined by the depth penetration mechanism. The interference fit between the lancet 20 and the collar C also ensures that substantial concentricity of the lancet 20 with the longitudinal axis 41 is maintained and the pointed end 24 of the lancet takes on the characteristic movement of the probe P, in terms of displacement, velocity and acceleration as described in the priority application Ser. No. 12/327,817. The clearances between the lancet body 26 and the collar 46 and that between the lancet body 27 and the bore 43 also ensure that the pointed end 24 of the lancet takes on the characteristic movement of the probe P, which is connected to a firing mechanism (not shown in FIG. 3).

In addition, when the lancet assembly 20 is fully inserted into the test meter 7, the sensing terminals T of the test meter 7 comes into contact with and ride on the terminal end 62, and a tongue or spigot Q at the receptacle R of the test meter 7 disengages or unlocks the L-shaped catch 32 from the end wall of the longitudinal slot 50 of the casing 40. In this unlocked position of the L-shaped catch 32, the L-shaped catch 32 and the entire lancet 20 is uninhibited in its movement but takes on the characteristic movement of the probe P when the firing mechanism of the probe P is activated. After firing of the probe, the probe P returns to its unprimed position, at which point the lancet 20 and the needle pointed end 24 are withdrawn into the casing 40. At the same time, the L-shaped catch 32 returns to its unlocked or disengaged position. To discard the used lancet device or assembly 10, the user pulls on the lancet casing 40 to free the entire lancet 10 device from the collar C whilst the L-shaped catch 32 is still disengaged. Once the lancet device 10 is being removed from the receptacle R, the L-shaped catch 32 springs back to its locked position and thereby locks the used lancet 20 inside the casing 40. The relocking of the used lancet 20 onto the casing 40 minimizes accidental pricking by the needle pointed end 24.

In one embodiment, the maximum projection of the needle pointed end 24 from the front face 42 of the casing 40 is given by the stroke S of the probe P minus L2. Depending on the skin characteristics at the intended blood sampling point, for example, thickness and hydration of the epidermis, the depth of wound puncture is a function of (S minus L2).

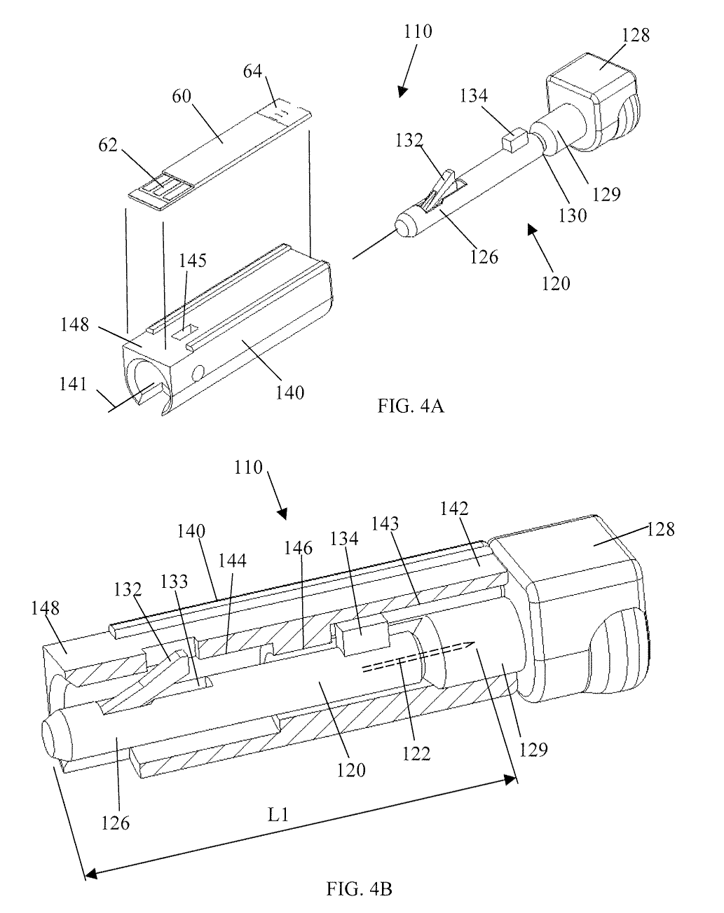

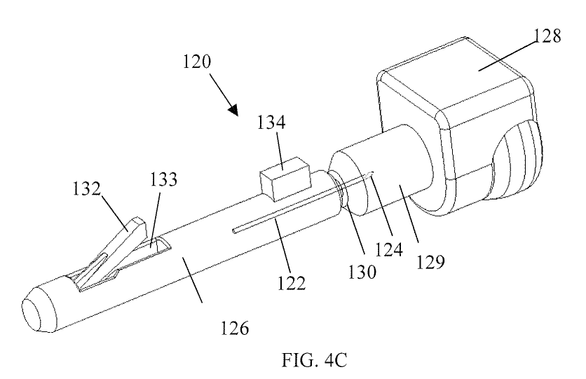

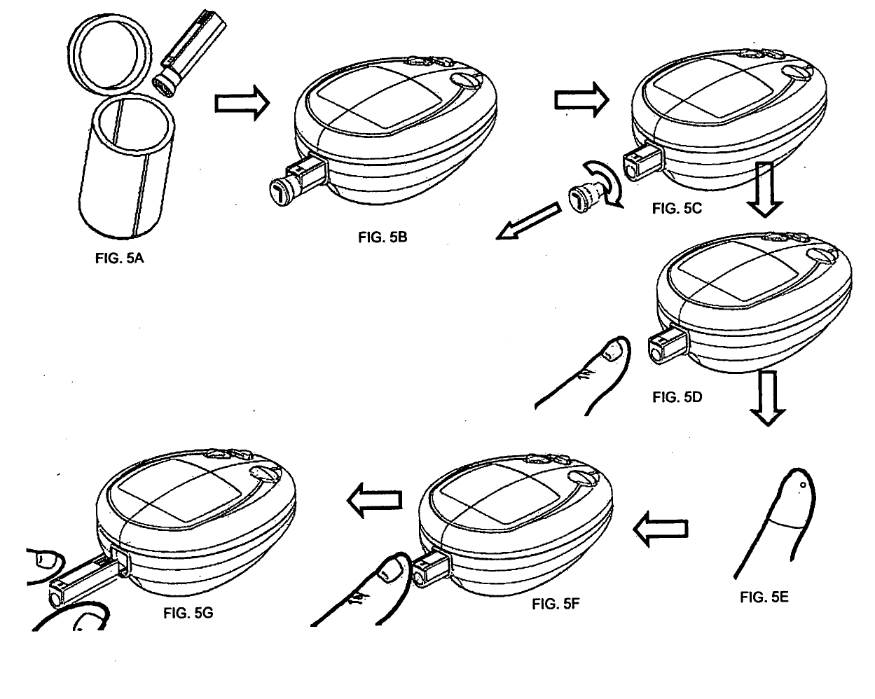

FIG. 4A shows a lancet device 110 according to another embodiment of the present invention. The lancet device 10 is an assembly of a lancet 120, a casing 140 and an analyte test strip 60. As shown in FIG. 5A, the lancet 120 is disposed inside the casing 140 and the test strip 60 is disposed on the casing 40. The lancet 120 includes a needle 122 that is sterilized and insert molded with a thermoplastic just like the earlier lancet 20. As shown in FIG. 4A, the needle pointed end 124 is molded within a cap 128. The cap 128 is connected to the molded lancet body 126 by a notch 130 of reduced cross-section. In between the cap 128 and the notch 130 is a locating part 129. The locating part 129 is cylindrical and concentric with the needle 122. On the lancet body 126 but near to the notch 130 is a stopper 134. The stopper 134 projects from the cylindrical surface of the lancet body 126. Also on the lancet body 126 but near to the free end of the lancet body 126 is a catch 132. The catch 132 is extended in its unactivated or locked position and lies on the same meridian as the stopper 134. The catch 132 is operable to deflect into its cavity 133 so that the catch 132 lies within the cylinder surface of the lancet body 126. Just like the earlier lancet 20, the length of the lancet 120 from the free end of the lancet body 126 to the needle pointed end 124 is L1.

FIG. 4B shows a sectional view of a lancet device 110 shown in FIG. 4A. The casing 140 is similar in length to the earlier casing 40. As in the earlier casing, the casing 140 is also hollow and has two cylindrical bores 143, 144. A collar 146 separates the two cylindrical bores 143,144. The cylindrical bore 143 is at the front end 142 of the casing 140 and has a slot 143a to accommodate the stopper 134. The cylindrical bore 43 is dimensioned to fit with the locating part 129 of the cap 128 to give an interference fit, whilst the fit between the stopper 134 and the slot 143a is clearance fit. The cylindrical bore 144 is dimensioned to accommodate the collar C of the probe P of the analyte test device 7 and the fit between the external dimension of the collar C and the cylindrical bore 144 is a clearance fit. The fit between the lancet body 126 and the collar 146 is also clearance fit. The cylindrical bore 144 has an aperture 145 that opens out to a top side 148 of the casing 140. The aperture 145 is dimensioned to accommodate the catch 132 when the lancet 120 is inserted into the casing 140 and the stopper 134 contacts the collar 146.

As in the previous embodiment, the fit between the collar C at the free end of the probe P and the lancet body 126 is an interference fit; this interference fit allows the lancet 120 to be retained in the probe P so that the lancet 120 and probe P move as one body during lancing of the probe P. In addition, this interference fit and the clearance fit around the lancet body 126 allow the lancet 120 to take on the characteristic movements of the probe P during lancing in terms of displacement, velocity and acceleration. In addition, the bottoming-out of the lancet 120 in the collar C allows a penetration depth of the lancet that is predetermined via a depth penetration mechanism (not shown in the figures) to be determinate.

When the lancet device 110 is inserted into the collar C of the probe P of the test meter 7, a lead-in chamfer at the collar C pushes the catch 132 down into its cavity 133 and unlocks the catch 132 from the aperture 145. In the earlier embodiment, when the lancet device 10 is inserted into the collar C, the lancet 20 is in contact with the test meter 7 through the spigot Q and the L-shaped catch 32. In this embodiment, when the lancet device 110 is inserted into the collar C, the lancet 120 does not contact any part of the test meter 7.

After firing of the probe, the probe P returns to its unprimed position and the lancet 120 are withdrawn into the casing 140. To discard the used lancet device 110, the user pulls on the lancet casing 140 to free the entire lancet device 110 from the collar C. Once the lancet device 110 is removed from the collar C, the catch 132 springs back to its unactivated or locked position and projects into the aperture 145 to lock the used lancet 120 inside the casing 140. Again, the relocking of the used lancet 120 inside the casing 140 minimises accidental pricking by the needle 122 and thus allows for the safe disposal of the used lancet device 110.

The stopper 134 shown in FIGS. 4A, 4B and 4C has a rectangular profile. In another embodiment of the lancet device 110, the stopper 134 has a substantially triangular profile such that a vertical face of the triangular profile contacts with a corresponding surface on the collar 146. In addition, the catch 132 and the stopper 134 need not lie on the same meridian on the cylindrical surface of the lancet body 126. In another embodiment of the lancet device 110, the stopper 134 is circular step like that of step 34 in the earlier embodiment. In another embodiment, it is possible that the aperture 145 lies on another face of the casing 140.

An advantage of providing the analyte test device 5, i.e. the lancet device 10,110 and associated analyte test meter 7, according to the present invention is that the number of steps in carrying out an analysis of an analyte in one's blood sample is fewer than those required for a conventional device. For example, in a conventional device, the steps involve in analyzing one's blood glucose level are as follow:
1. Removing a new lancet from its container;
2. removing a cover on a lancing device;
3. inserting the new lancet into the lancing device;
4. removing the lancet safety cap;
5. putting back the cover onto the lancing device;
6. priming the lancing device;
7. removing a new test strip from its container;
8. inserting the new test strip on a test meter;
9. lancing a sampling area with the lancing device to make a skin puncture;
10. allowing a droplet of blood to ooze out from the skin puncture;
11. applying the blood sample onto the test strip and obtaining a reading on the test meter;
12. discarding the used test strip from the test meter;
13. removing the cover from the lancing device;
14. putting back the safety cap onto the used lancet;
15. removing the used lancet from the lancing device; and
16. replacing the cover onto the lancing device.
   In contrast, in the present invention, the number of steps required to conduct an analysis of one's blood sample, as shown in FIGS. 5A-5G, have accordingly been reduced to seven steps as follow:
1a. removing a lancet device 10,110 that has an integral test strip 60 from its container;
1b. inserting the lancet device 10,110 into an associated test meter;
1c. tearing away the protective cap 28,128 from the lancet device 10,110;
1d. priming and firing the lancet mechanism in the test meter to puncture one's skin;
1e. allowing a droplet of blood to ooze from the skin puncture;
1f. transferring the droplet of blood onto the sensing end of the test strip and allowing the test meter to generate a reading; and
1g. after the test is completed, removing the lancet device 10,110 from the test meter for disposal.
   Although the number of steps have been reduced, there is no substantive change that a user has to learn in using the analyte test device of the present invention.

Another advantage of the present invention includes relocking of a used lancet 20,120 inside the casing 40,140 so that the used lancet device 10,110 can be disposed off in a safe manner. When using a conventional lancing device, the skin puncture point is close to the lancet cover and periodic cleaning of blood stains is necessary. With the present invention, the relative distance from the skin puncture point and test meter 7 is greater than the skin puncture point to the conventional lancing device, so there is little likelihood of blood stain on the test meter; thus, there is no need for periodic cleaning to remove blood stains off the test device. In addition, as the lancing mechanism is integrated within the test meter 7, there is no additional cleaning of a separate lancing device; if there is blood stain, it is likely to appear on the used lancet devices 10,110, which are disposed off. The unused lancet devices 10,110, together with the test strips 60, are kept in air-tight container in compliance with the manufacturer's directions so that reliability of the test strips is maintained.

Another advantage of the present invention is that it allows a user control over the individual steps in collecting a blood sample. For example, a user may be used to milking one's finger to ooze out a droplet of blood. The user of the present invention is able to do so after firing the lancing mechanism in the test meter 7; once a sufficient amount of blood has been oozed out, the blood droplet is transferred onto the analyte sensing end 64 on the test strip 60. In the event of a user not being able to obtain a sufficient amount of blood when using some known diagnostic devices, for example a fully automatic diagnostic device, the device has to be primed again to make another skin puncture and often resulting in a test strip being wasted; instead, with the present invention, the user can milk one's finger to obtain a sufficient amount of blood or re-prime the lancing mechanism to make another skin puncture, albeit deeper penetration, without wasting the lancet device 10,110 that has been inserted into the test meter 7.

While specific embodiments have been described and illustrated, it is understood that many changes, modifications, variations and combinations thereof could be made to the present invention without departing from the scope of the invention. For example, whilst testing of blood glucose is envisaged, the present invention is not so limited; rather, the test device and method of the present invention is also possible for testing blood cholesterol, uric acid, ketone, etc. and specific chemicals in other physiologic fluids like interstitial fluid, urine, sweat, saliva, and so on.

What is claimed is:

1. An analyte test device for testing a physiologic fluid, the device comprising:
a test meter; and
a lancet device having a casing, a lancet and a test strip; wherein the lancet is disposed and locked inside the casing by a catch, the test strip is disposed on said casing; and said lancet device is operable to be removeably connected to said test meter via a receptacle on said test meter;
wherein said receptacle is operable to engage with said catch to unlock said lancet from said casing when said lancet device is inserted into said receptacle and to disengage said catch to relock said lancet inside said casing when said lancet device being removed from said receptacle.

2. An analyte test device according to claim 1, wherein said receptacle comprises a spigot, which cooperates with said catch to unlock and lock the lancet inside said casing.

3. An analyte test device according to claim 2, further comprising a collar at a probe, which is coupled to a lancet firing mechanism inside the test meter, and said lancet is operable to be inserted into said collar.

4. An analyte test device according to claim 3, wherein said lancet is inserted into said collar with an interference fit so that the lancet is operable to take on the characteristic of the lancet firing mechanism.

5. An analyte test device according to claim 1, wherein said receptacle comprises a collar at a probe, which is coupled to a lancet firing mechanism inside the test meter, and said lancet is operable to be inserted into said collar.

6. An analyte test device according to claim 5, wherein said lancet is inserted into said collar with an interference fit so that the lancet is operable to take on the characteristic of the lancet firing mechanism.

7. An analyte test device according to claim 1, wherein said lancet is inserted into said casing with a clearance fit.

8. An analyte test device according to claim 1, wherein said lancet has a pointed end, which is sterilized and insert molded but protected by a breakable cap.

9. A kit comprising:
a lancet;
a lancet casing; and
a test strip;
wherein said lancet has a catch, said catch is operable to lock said lancet inside said lancet casing, and said test strip is operable to be attached onto said lancet casing to form a lancet device.

10. A kit according to claim 9, further comprising a test meter, said test meter having a receptacle for receiving said lancet device.

11. A method for determining an analyte in a physiologic fluid, said method comprising:
supplying a lancet device, said lancet device comprises a lancet, a lancet casing and a test strip; wherein said lancet has a catch that is operable to lock said lancet inside said lancet casing, and said test strip is operable to be attached onto said lancet casing to form a lancet device;
inserting the lancet device into a receptacle of a test meter, wherein said catch is then deactivated and the lancet is operable to take on the characteristic of a lancet firing mechanism disposed inside said test meter;
collecting a physiologic fluid sample and transferring the fluid sample on an analyte sensing end on said test strip; and
obtaining a reading from said test meter.
